(19) United States
(12) Patent Application Publication
(10) Pub. No.: US 2012/0289819 A1
(43) Pub. Date: Nov. 15, 2012

(54) IMPLANT DETECTOR

(75) Inventor: Sean Snow, Carpinteria, CA (US)

(73) Assignee: ALLERGAN, INC., Irvine, CA (US)

(21) Appl. No.: 13/103,767

(22) Filed: May 9, 2011

(51) Int. Cl. A61B 5/05; A61F 2/00

(52) U.S. Cl. 600/424; 600/37

(57) ABSTRACT

Generally described herein are an apparatus, a method of use, and/or a system related to an implant detector for detecting whether a needle inserted into a body of a patient has come into contact with a sensitive portion of the implanted gastric banding system (e.g., the tube connecting the access port to the gastric band). For example, a warning light may be activated. By alerting the medical professional when the inserted needle has come into contact with a sensitive portion of the implanted gastric banding system, the implant detector may prevent the medical professional from puncturing the contacted sensitive portion.

### FIELD

The present invention generally relates to medical systems and apparatus and uses thereof for treating obesity and/or obesity-related diseases, and more specifically, relates to, for example, a system for detecting whether a needle inserted into a body of a patient has come into contact with or in close proximity to a sensitive portion of the implanted gastric banding system (e.g., the tube connecting the access port to the gastric band).

### BACKGROUND

Adjustable gastric banding apparatus have provided an effective and substantially less invasive alternative to gastric bypass surgery and other conventional surgical weight loss procedures. Unlike gastric bypass procedures, gastric band apparatuses are reversible and require no permanent modification to the gastrointestinal tract. Moreover, it has been recognized that sustained weight loss can be achieved through a laparoscopically-placed gastric band, for example, the LAP-BAND® (Allergan, Inc., Irvine, Calif.) gastric band or the LAP-BAND AP® (Allergan, Inc., Irvine, Calif.) gastric band. Generally, gastric bands are placed about the cardia, or upper portion, of a patient's stomach forming a stoma that restricts food's passage into a lower portion of the stomach. When the stoma is of an appropriate size that is restricted by a gastric band, food held in the upper portion of the stomach may provide a feeling of satiety or fullness that discourages overeating. An example of a gastric banding system is disclosed in Roslin, et al., U.S. Patent Pub. No. 2006/0235448, the entire disclosure of which is incorporated herein by this specific reference.

Over time, a stoma created by a gastric band may need adjustment in order to maintain an appropriate size, which is neither too restrictive nor too passive. Accordingly, prior art gastric band systems provide a subcutaneous fluid access port connected to an expandable or inflatable portion of the gastric band. By adding fluid to or removing fluid from the inflatable portion by means of a hypodermic needle inserted into the access port, the effective size of the gastric band can be adjusted to provide a tighter or looser constriction.

However, when a medical professional attempts to insert the hypodermic needle into the body of the patient, it may be difficult to know where the tip of the needle is. As a result, the needle may unintentionally pierce and/or cause damage to the soft components of the implanted medical device during an attempt to insert the needle into the access port.

Some attempts have been made to assist the medical professional in improving the accuracy of the needle insertion procedure. For example, with reference to FIG. 1, Steinbeck, U.S. Pat. No. 5,649,546 discloses a wand external to the body of the patient which produces electromagnetic fields and detects changes to that field caused by the presence of metal objects. However, the system of Steinbeck does not allow the medical professional to determine if the needle has come in contact with a soft component and/or a sensitive portion of the implanted gastric banding system (e.g., the tube connecting the access port to the gastric band).

With reference to FIG. 2, Gentelia et al., U.S. Pat. No. 5,496,313 discloses a system which allows the medical professional to determine when the needle penetrates a bodily membrane, but does not assist the medical professional in determining when the needle has come in contact with the soft component and/or the sensitive portion of the implanted gastric band.

With reference to FIG. 3, Sampson et al., U.S. Pat. No. 4,286,584 discloses an access port containing a permanent magnet and an external device which detects the magnetic field caused by the magnet, but again does not utilize a needle which enters the body, and further does not allow the medical professional to determine if the needle has come in contact with the soft component and/or sensitive portion of the implanted gastric band.

Accordingly, it is desirable to develop an implant detector which signals or warns a medical professional when a hypodermic needle comes into contact with or in close proximity to the soft component and/or sensitive portion of the implanted gastric band.

### SUMMARY

Generally described herein include an apparatus, a method of use, and/or a system related to an implant detector for detecting whether a needle inserted into a body of a patient has come into contact with a sensitive portion of the implanted gastric banding system (e.g., the tube connecting the access port or the injection port to the gastric band). By alerting the medical professional when the inserted needle has come into contact with a sensitive portion of the implanted gastric banding system, the implant detector may prevent the medical professional from puncturing the contacted sensitive portion.

In one embodiment, the present invention is an implant detection system for detecting whether a needle is in contact with a conductive component of a gastric banding system for the treatment of obesity and/or obesity-related diseases, the implant detection system comprising (1) a gastric band having an inflatable portion disposed about a esophageal-gastric junction of a patient, (2) a tubing having an exterior surface and fluidly coupled to the inflatable portion of the gastric band at a first end, (3) a conductive film covering the exterior surface of the tubing, the conductive film serving as the conductive component, (4) an access port having a septum, the access port coupled to the tubing at a position located at a second end of the tubing, and for the addition or removal of fluid from the inflatable portion of the gastric band via the tubing, (5) a syringe having a plunger and a barrel, the syringe for storing the fluid to be added or the fluid removed, (6) a needle having a base and a tip, the base of the needle coupled to the barrel of the syringe, the needle further having a channel for carrying the fluid between the base and the tip, and (7) an electronic indicator coupled to an outside surface of the needle, such that when the tip of the needle contacts the conductive film of the tubing, the electronic indicator is activated.

In one embodiment, the present invention is an implant detector including a standard syringe having an electronic unit and a needle, a return electrode, and a modified access port and tubing. When the needle is inserted by the medical professional attempting to intentionally penetrate the septum of the access port (to add or remove fluid from the implanted gastric band), the electronic unit or signal processor may produce a small, physiologically-safe electrical signal and may measure the return signal. If the needle comes into contact with an area of the access port or tubing susceptible to unintended needle puncturing, then the return signal may be altered which may cause a warning (e.g., activation of a warning light) to be issued to the medical professional. In this manner, the medical professional may be properly warned that continuing to insert the needle in the current trajectory may puncture the susceptible area. The return electrode may be placed on the exterior of the patient's skin, and may serve to test the functionality of the implant detector before the needle is inserted into the patient. For example, by touching the needle to the return electrode, the warning may be issued to the medical professional, thereby confirming that the implant detector is working properly.

In one embodiment, the electronic unit may be reconfigured such that if the needle comes into contact with an area of the access port or tubing susceptible to unintended needle puncturing, then the return signal may be altered which may cause a warning (e.g., deactivation of an “okay” light) to be issued to the medical professional. In this manner, the switching off of the “okay” light may alert the medical professional to stop inserting the needle in the current trajectory or risk puncturing the susceptible area.

In one embodiment, the return electrode may be incorporated into the design of the hypodermic needle such that the needle is a bipolar electrode instead of a monopolar electrode.

### BRIEF DESCRIPTION OF THE DRAWINGS

FIG. 1 illustrates a prior art system that utilizes a wand external to the body of the patient which produces electromagnetic fields and detects changes to that field caused by the presence of metal objects.

FIG. 2 illustrates a prior art system which allows the medical professional to determine when the needle penetrates a bodily membrane.

FIG. 3 illustrates a prior art access port containing a permanent magnet and an external device which detects the magnetic field caused by the magnet.

FIG. 4 illustrates a perspective view of the implant detector and an implanted gastric banding system according to an embodiment of the present invention.

FIG. 5 illustrates a perspective view of the implant detector with an activated light in contact with a tubing of the gastric banding system according to an embodiment of the present invention.

FIG. 6 illustrates a perspective view of the implant detector with a deactivated light in contact with a septum of the gastric banding system according to an embodiment of the present invention.

FIG. 7 is a flowchart of a method of operating the implant detector according to an embodiment of the present invention.

FIG. 8A is an exploded view of an implant detector according to an embodiment of the present invention.

FIG. 8B is a cross-sectional view of the implant detector of FIG. 8A according to an embodiment of the present invention.

FIG. 9 is a circuit diagram of the detection circuit for an implant detector according to an embodiment of the present invention.

FIG. 10A is a perspective view of the access port and tubing according to an embodiment of the present invention.

FIG. 10B is a cross-sectional view of the access port and tubing of FIG. 10A according to an embodiment of the present invention.

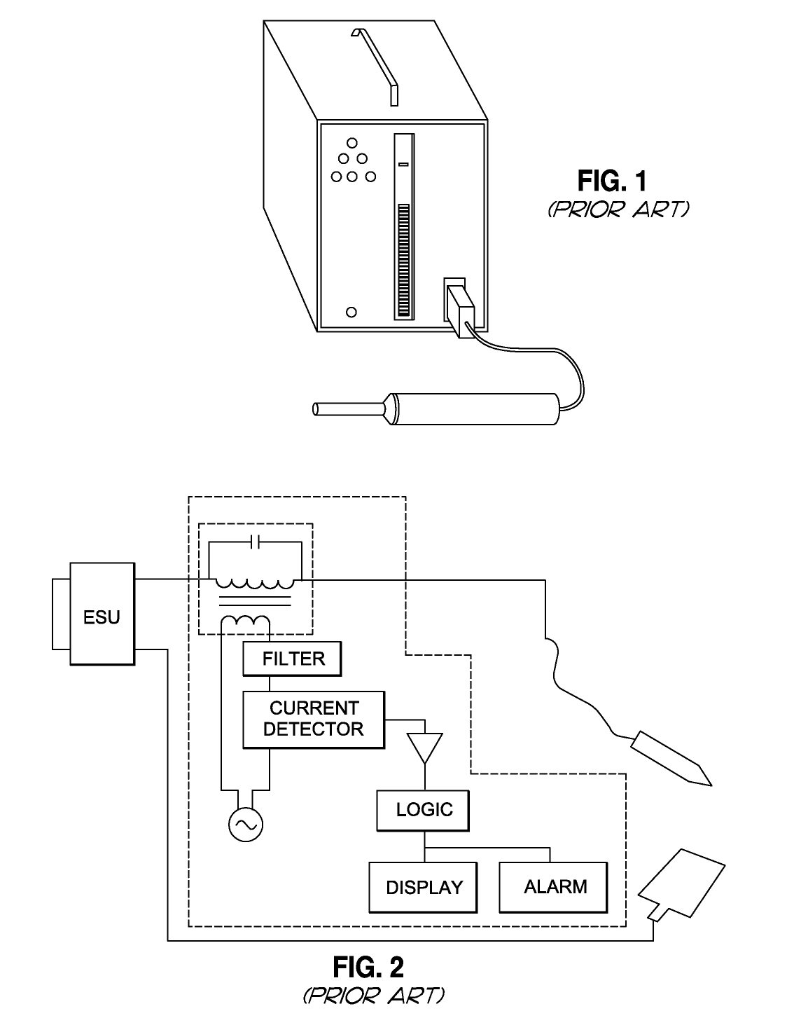

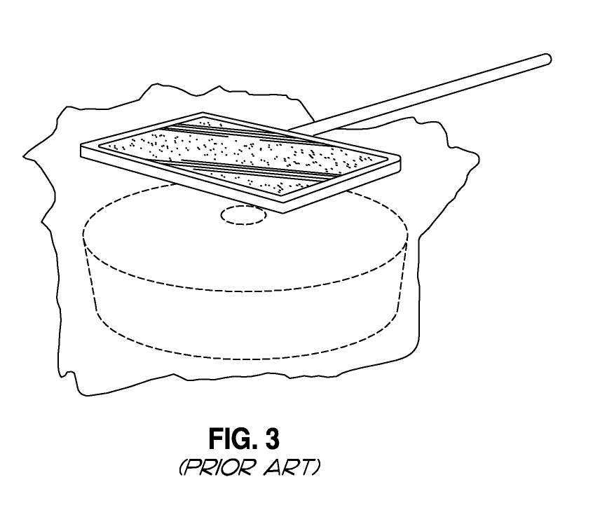

### DETAILED DESCRIPTION

The present invention generally provides an apparatus, a method of use, and/or a system for detecting whether a needle inserted into a body of a patient has come into contact with a sensitive portion of the implanted gastric banding system (e.g., the tube connecting the access port to the gastric band) and issuing a warning (e.g., a light being activated) in response to detecting that the needle has come into contact with the sensitive portion.

Generally, the apparatus or system does not require any changes to the normal implantation procedure of the gastric banding system. After a period of time (e.g., a few weeks) after the gastric banding system has been implanted, as deemed appropriate by a medical professional, a patient may report or request for an adjustment to the volume of the fluid within the gastric banding system. For example, fluid may be added if the gastric band needs to be tightened or fluid may be removed if the gastric band needs to be loosened.

In one embodiment, a medical professional may connect a syringe to the modified needle and assuming that the electronic device is not attached yet to the syringe or needle, attach the electronic device over a portion of the needle to place it in the proper position near and/or over a hub of the needle. A return electrode connected to the syringe or the electronic device may be placed on the patient's skin near the abdomen area. The electronic device may be switched on and tested by lightly touching the tip of the hypodermic needle to a metal portion of the return electrode (e.g., a metal tab). If the warning light is activated when the needle is touched to the return electrode, the needle is functional and can be inserted into the patient in an attempt to penetrate the septum of the access port.

If the medical professional is successful in penetrating the septum of the access port, the warning light will remain deactivated, and the medical professional may enter the access port and perform the gastric banding system adjustment. However, if the needle contacts an unintended, sensitive portion of the gastric banding system (e.g., the tubing) covered by a conductive film, the warning light is activated or illuminated and the medical professional is alerted in this manner not to continue to insert the needle as it may puncture the sensitive portion.

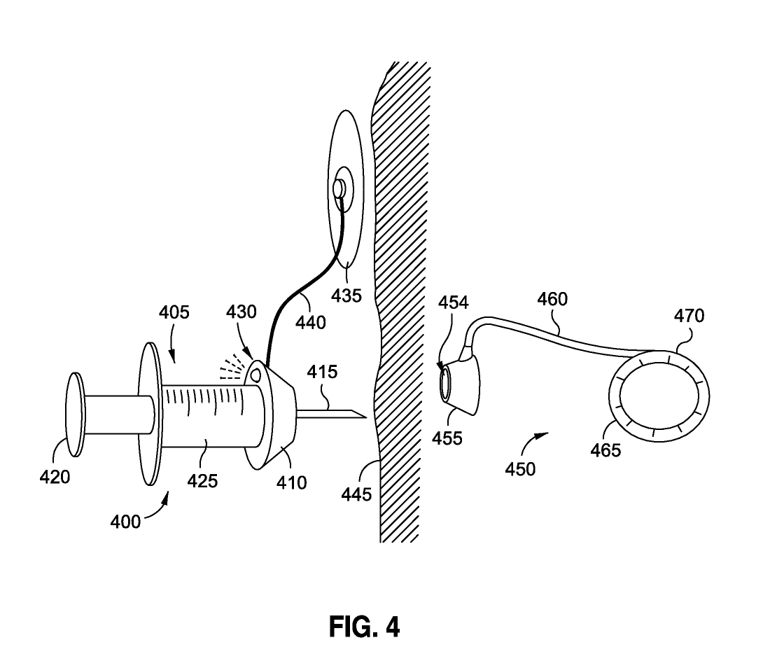

Turning now to FIG. 4, an embodiment of an operational set-up of an implant detector 400 is shown. The implant detector 400 may include a syringe 405 having a plunger 420 and a barrel 425. The syringe 405 may be connected to a needle 415 with an electronic device or signal processor 410 attached at a location proximal to the connection point of the syringe 405 and the needle 415. The electronic device 410 may be attached to a return electrode 435 via a tether 440. The return electrode 435 may be placed and/or adhered to a patient's skin 445 proximal to the abdomen area of the patient, thereby allowing the needle 415 to be insertable through the patient's skin and reach an access port 455 of an implanted gastric banding system 450. The gastric banding system 450 may include the access port 455 having a septum 454, a gastric band 465 having an inflatable portion 470 and a tube 460 fluidly connecting the access port to the inflatable portion 470 of the gastric band 465. Other portions and/or details of the gastric banding system 465 have been omitted from FIG. 4 for clarity.

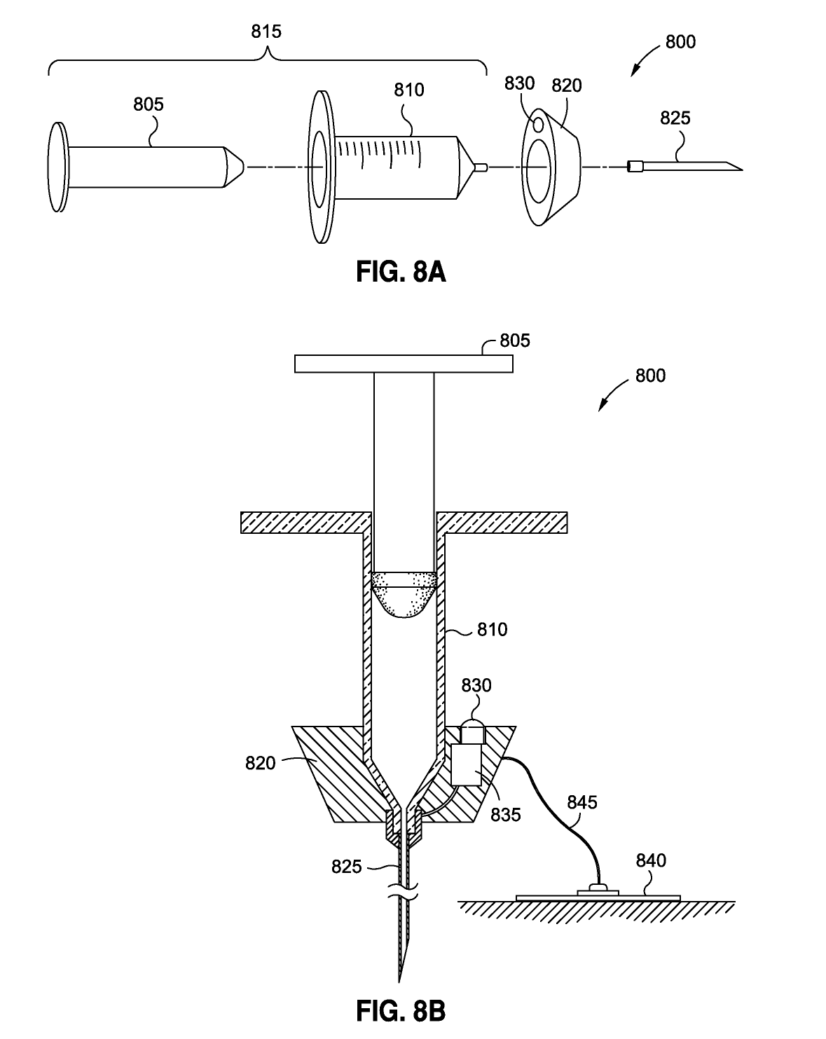

The return electrode 435 may function as a testing unit to ensure that the electronic device 410 is functioning properly before the medical professional proceeds to insert the needle 415 through the patient's skin 445 in an attempt to locate the septum 454 to add or remove fluid from the gastric banding system 450. When the electronic device 410 is operating properly, a light or electronic indicator 430 of the electronic device 410 may be activated when the tip of the needle 415 makes contact with the return electrode 435. Alternatively, or in addition, other testing methods may include touching the shank of the needle 415 or other test zone to the return electrode 435, sliding a test device over the outside of the guard of the needle 415 to couple capacitively to the return electrode current path, and the like. More details related to how the return electrode 435 causes the light 430 of the electronic device 410 to be activated is discussed below with respect to FIG. 8B.

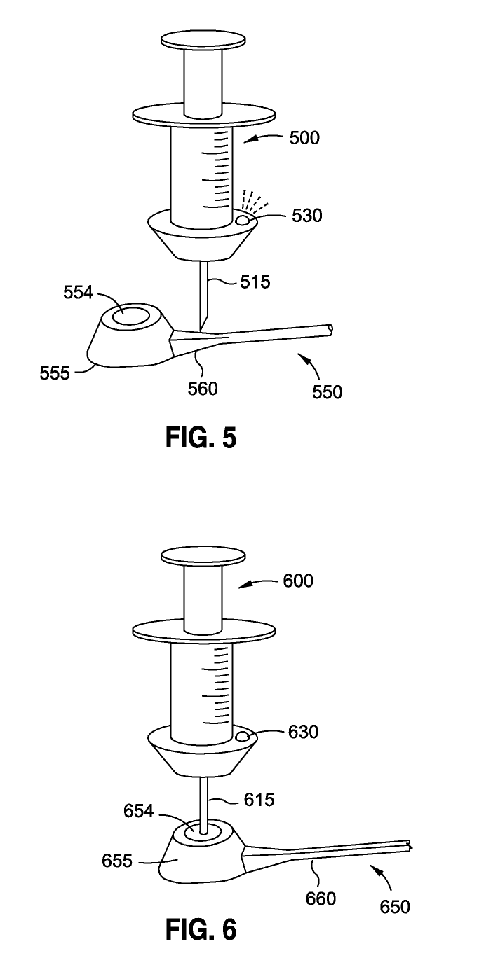

FIG. 5 illustrates an implant detector 500 with a needle 515 in contact with an outer surface of a tubing 560 with certain portions of the implant detector 500 and a gastric banding system 550 omitted for clarity. As shown, a light 530 on the implant detector 500 may be illuminated or activated when the needle 515 comes into contact with the outer surface of the tubing 560 signaling or alerting the medical professional not to further insert the needle 515 as doing so may puncture the tubing 560. By alerting the medical professional that the needle 515 is in contact with the tubing 560, the medical professional may be able to retract the needle 515 prior to puncturing the tubing 560 and redirect the needle 515 in a proper direction towards a septum 554 of an access port 555. In this manner, the light 530 may serve two functions: first, warning that further insertion of the needle 515 may puncture the tubing 560 and second, provide a general idea of the direction that the medical professional may have to adjust the needle towards in order to find the septum 554 since the medical professional will know of the orientation between the access port 555 and the tubing 560.

Alternatively, in one embodiment, the light 530 may be activated even when the needle 515 does not actually physically contact the tubing 560. For instance, if the needle 515 is within a predetermined distance away (e.g., between about 0.001 millimeters to about 1 centimeter) from the surface of the tubing 560, thereby indicating that the needle 515 is not positioned correctly and may be on the verge of puncturing the tubing 515, the light 530 may be activated. More particularly, the resulting capacitance shift caused by being proximate to the conductive material on the tubing 560 may be detected by the needle 515 and activation of the light 530 may be triggered.

In one embodiment, the light 530 may be configured to be activated when the needle 515 is within a 0.001 millimeter envelope or radius of the outer surface of the tubing 560.

FIG. 6 illustrates an implant detector 600 with a needle 615 inserted into a septum 654 with certain portions of the implant detector 600 and gastric banding system 650 omitted for clarity. As shown, a light 630 on the implant detector 600 might not be illuminated and/or remains deactivated when the needle 615 comes into contact and penetrates the septum 654 of an access port 655, thereby allowing the medical professional to confidently proceed with the insertion of the needle 615 without worrying whether a tubing 660 will be punctured by the needle 615. As shown in FIG. 6, the needle 615 is properly inserted into the septum 654 and in this configuration, the medical professional may begin to make the fluid adjustment to the gastric banding system 650.

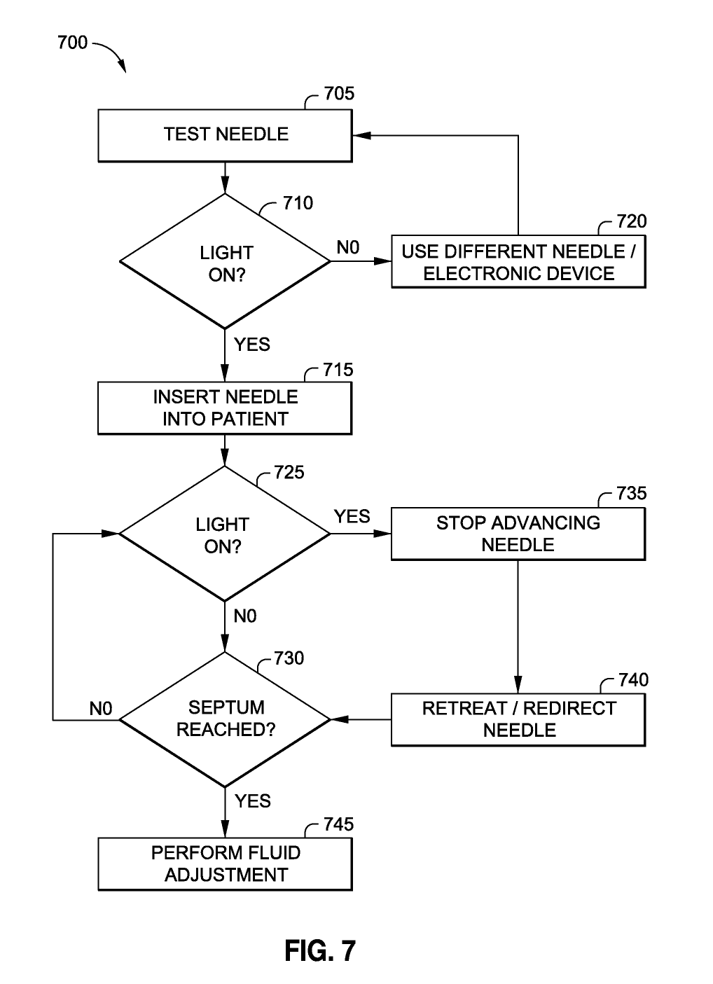

FIG. 7 illustrates a flowchart 700 depicting a method of operation of an implant detector (e.g., the implant detector 400, 500, 600) with respect to a gastric banding system (e.g., the gastric banding system 450, 550, 650) during a fluid adjustment procedure. While this method may be followed with the use of any of the above described implant detectors, reference will be made with respect to the implant detector 400 of FIG. 4 for simplicity. At step 705, a medical professional may turn on the electronic device 410 and may test the electronic device 410 by touching the needle 415 to the return electrode 435. At step 710, if the light 430 activates when the needle 415 is in contact with the return electrode 435 and deactivates when the needle 415 is no longer in contact with the return electrode 435, the electronic device 410 may be functioning properly and the process may move to step 715. However, if the light 430 fails to activate when the needle 415 is in contact with the return electrode 435, the implant detector 400 may be deemed dysfunctional, and the process may move to step 720 where a replacement implant device may be obtained, and the testing process may be restarted.

At step 715, after the implant detector 400 is deemed functional, the needle 415 may be inserted through the patient's skin in an attempt to find the septum 454. At step 725, the medical professional may check the light 430 on the implant detector 400 to ascertain whether the light 430 is activated or deactivated. If the light 430 remains deactivated, at step 730, the medical professional may continue to insert the needle 415 deeper into the tissue of the patient until the septum 454 is penetrated by the needle 415, and at which point, the fluid adjustment may be performed by the medical professional at step 745. However, if at any point during needle insertion, the light 430 becomes activated, the process may move from step 725 to step 735 where the advancement of the needle 415 should be promptly stopped, as activation of the light 430 serves as a warning to the medical professional that the needle 415 is in contact with the tubing 460 and that further insertion of the needle 415 may result in unintended puncturing of the tubing 460. At step 740, the medical professional may retract the needle 415 and change a direction of the needle 415 in search of the septum 454. As the medical professional may be aware of the spatial relationship between the septum 454 and the tubing 460, activation of the light 430 may serve to provide directional assistance to the medical professional as to where the septum 454 may be located. After redirecting the needle 415 at step 740, the process may move to step 730. In this manner, the medical professional may be able to more effectively insert the needle 415 into the septum 454 without puncturing the tubing 460.

FIG. 8A depicts an exploded view of an implant detector 800. In one embodiment, the implant detector 800 may be the implant detector 400, 500, 600. As shown, the implant detector 800 may comprise four main components: a plunger 805 and a barrel 810 constituting a syringe 815, an electronic device 820 and a needle 825. The syringe 815 may function to add or remove fluid from a gastric banding system when the needle 825 is inserted into an access port of the gastric banding system thereby establishing a fluid path between the syringe 815 and the gastric banding system. In one embodiment, the needle 825 may be a non-coring, hypodermic needle and may include a thin layer of insulator on the needle shank. The insulation may improve the discrimination of the electronics and may improve the overall system performance. However, the electronic device 820 may also be compatible with standard needles without insulation, thereby providing retrofit functionality to existing needles.

As is typical, the barrel 810 may act as a reservoir for holding the fluid while the plunger 805 may act to either pull fluid into the barrel 810 from the gastric banding system or act to push fluid from the barrel 810 into the gastric banding system via the needle 825. Attached or otherwise held in place between the barrel 810 and the needle 825 is the electronic device 820. In one embodiment, the electronic device 820 includes a housing with one or more electronic components contained therein. The housing may be sealed to prevent fluid from coming into contact with the electronic components. The electronic device 820 further includes a light or display 830, and is not in fluid communication with the syringe 815 or the needle 825. However, the electronic device 820 may be electrically coupled to the needle 825 such that an electrical pulse or signal generated by the electronic device 820 may travel through a conductive portion of the needle 825 and a return signal based on the generated electrical pulse or signal may travel back through the electronic device 820 and back to the electronic device 820 when the needle 825 is, for instance, in contact with another conductive material (e.g., a conductive coating on the tubing 460 of FIG. 4). In one embodiment, the electrical device 820 may include a power switch (not shown) and a second light (not shown) for indicating whether the electronic device 820 is powered on or off.

FIG. 8B illustrates a cross-sectional view of the implant detector 800 of FIG. 8A in an assembled configuration. The electrical device 800 is shown here to include a control circuit 835 electrically connected with the needle 825 and the light 830. The return electrode 840, which acts as a test pad, is also shown tethered to the electrical device 820 via a flexible cord 845. The control circuit 835 may function to power the light 830 and provide a visual indication to the medical professional whether further insertion of the needle 825 is safe. While the return electrode 840 illustrated here in FIG. 8B may be a surface electrode similar to those used for electrocardiograms and/or electroencephalograms, different embodiments of the return electrode 840 may be possible. For example, in one embodiment (not shown), the return electrode 840 may be designed to be incorporated into the needle 825 (thereby obviating the need for the flexible cord 845). In this embodiment, the needle 825 may be a bipolar electrode instead of a monopolar electrode. In another embodiment, additional return electrodes, such as needle electrodes, may be utilized.

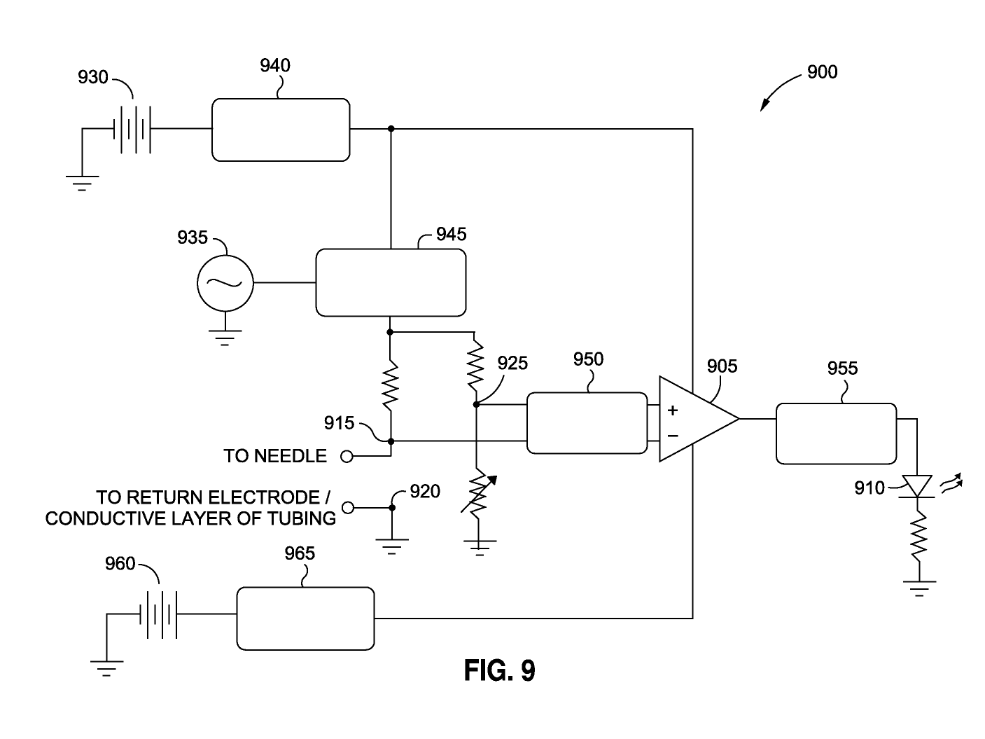

FIG. 9 illustrates a circuit 900 which, in one embodiment, may be implemented as the control circuit 835 of FIG. 8B. In essence, the circuit 900 includes a comparator 905 which outputs a current that powers a warning light 910 (e.g., a light emitting diode) when a node 915 (of the needle 825) is grounded (e.g., when the needle 825 is in contact with a node 920 which may correspond to the return electrode 840 and/or the conductive layer of the tubing). Otherwise, when the node 915 is not in contact with the node 920, the voltage at the node 915 is equal to the voltage at a node 925 and the comparator 905 does not output a current to drive the warning light 910. In this manner, nodes 915 and 920 act as a “switch”: when the nodes 915 and 920 are electrically connected (e.g., in direct contact with each other), the warning light 910 is activated; otherwise when nodes 915 and 920 are not electrically connected (e.g., when not in direct contact with each other), the warning light is deactivated. As shown in FIG. 9, other circuit elements including a battery 930, a waveform generator 935, a voltage regulator 940, an amplifier 945 may be included. In addition, in one embodiment, optional filters 950, 955 and an optional battery 960 and a voltage regulator 965 may also be utilized.

The electric excitation source (e.g., the battery 930) may control the voltage and/or current. In addition, in one embodiment, the charge flux may be balanced, while in another embodiment, the charge flux may be unbalanced.

The waveform generator 935 may generate one or more waveforms for driving current flow between the needle 825 and the return electrode 840. For example, a biphasic, sinusoidal waveform may be generated. Alternatively and/or in addition, a monophasic waveform, a square waveform, a rectangular waveform, a trapezoidal waveform or any other waveform, including custom-designed waveforms may be utilized.

As discussed thus far, the light 830 is a warning light or display which activates to warn the medical professional when the needle 825 is in contact with the tubing (not shown). However, in one embodiment, the light 830 may be configured to be an “okay” light or where an activation of the light 830 may indicate that it is safe to continue to insert the needle 825. However, such an embodiment may require modification to the circuit 900 such that, for example, the comparator 905 is inverted and outputs a current that powers the warning light 910 when the node 915 is not in contact with the node 920, and the voltage at node 915 is equal to the voltage at node 925. Otherwise, when the node 915 (of the needle 825) is grounded (e.g., when the needle 825 is in contact with node 920 corresponding to the return electrode 840 and/or the conductive layer of the tubing) the light 830 is deactivated. One skilled in the art will understand that further configurations are also possible by, for example, adding a conductive layer to the septum instead of the tubing.

In addition to, or as an alternative to the light 830, other feedback mechanisms may be used to provide information as to the location of the needle 825. For example, multiple, different colored lights may be implemented (e.g., a green light may indicate “continue insertion”, a yellow light may indicate that the tip of the needle 825 is in contact with a non-bodily tissue or material, and a red light may indicate “stop” or that the tip of the needle 825 is in contact with the conductive portions of the tubing). Alternatively, or in addition, an optical display (e.g., LCD) may provide text corresponding to the status of the needle 825. Other forms of feedback may include auditory feedback (e.g., audio messages), tactile feedback and/or other sensory feedback.

In one embodiment, the circuit 900 as described herein, may be configured to produce an alternating voltage. However, alternatively or in addition, the implanted layer of conductive metal on the tubing may be configured to create a small voltage, and when the needle (e.g., having a metal different than the conductive metal of the tubing) comes into contact with the conductive metal of the tubing, the small voltage created may be detected by the electronic device.

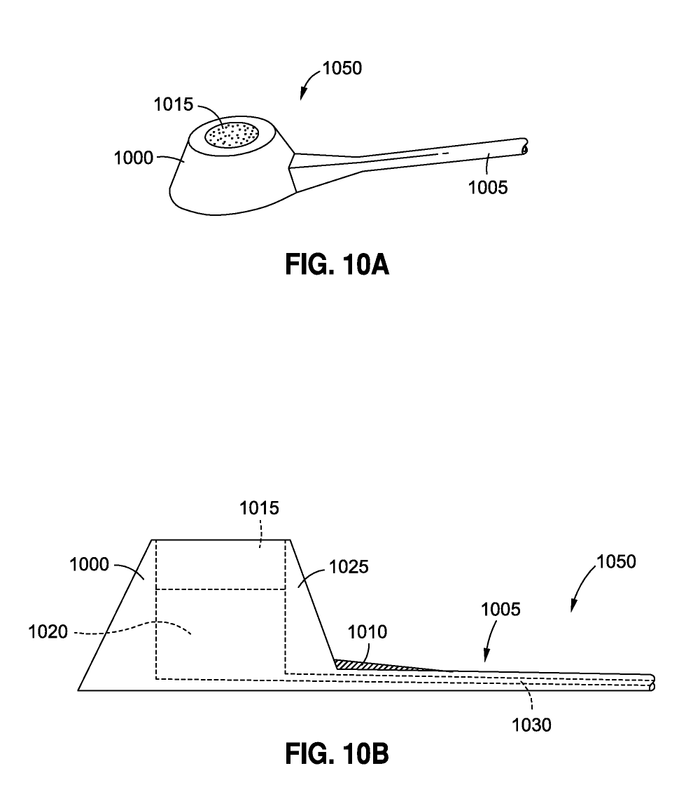

FIG. 10A illustrates a perspective view of an access port 1000 having a septum 1015, with an attached tubing 1005 of a gastric banding system 1050. Certain other portions of the gastric banding system 1050 have been omitted for clarity. The tubing 1005 may include a conductive coating (e.g., a bulk metal) which is exposed to the patient's body such as gold. Alternatively, or in addition, any conductive, biocompatible material may be used such as platinum, titanium, chromium, indium-titanium oxide, stainless steel, tungsten, nitinol and/or any combinations thereof. The conductive coating may be a metal film of varying thicknesses. For example, the conductive coating may be a thin film between about 50-50,000 angstroms thick and may wrap about the entire outer surface of the tubing 1005. In one embodiment the thickness of the conductive coating may be adjusted to result in a resistivity which permits compatibility with magnetic resonance imaging (MRI) procedures. Additionally, and/or alternatively, the length of tubing 1005 may be adjusted to permit compatibility with MRI procedures.

FIG. 10B illustrates a cross-sectional view of the access port 1000 and the tubing 1005 of the gastric banding system 1050 of FIG. 10A. As shown here, the access port 1000 may include a housing 1025 which defines a cavity for a septum 1015 and a cavity for fluid 1020, adjacent to the cavity for the septum 1015, for insertion or removal of fluid within the gastric banding system 1050. The cavity 1020 may be fluidly connected to a tubing cavity 1030 of tubing 1005, thereby establishing a fluid path between the cavity 1020 and the rest of the gastric banding system 1050. The tubing 1005 may also include a conductive layer 1010 which, in one embodiment, may increase in thickness closer to the access port 1000. Alternatively, the thickness of the conductive layer 1010 may be uniform. In one embodiment, the access port 1000 may also include conductive portions (not shown) on the outer surface that functional substantially the same as the conductive layer 1010.

Unless otherwise indicated, all numbers expressing quantities of ingredients, volumes of fluids, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, certain references have been made to patents and printed publications throughout this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An implant detection system for detecting whether a needle is in contact with a conductive component of a gastric banding system for the treatment of obesity and/or obesity-related diseases, the implant detection system comprising:
a gastric band having an inflatable portion disposed about a stomach of a patient;
a tubing having an exterior surface and fluidly coupled to the inflatable portion of the gastric band at a first end;
a conductive film covering the exterior surface of the tubing, the conductive film serving as the conductive component;
an access port having a septum, the access port coupled to the tubing at a position located at a second end of the tubing, and for the addition or removal of fluid from the inflatable portion of the gastric band via the tubing;
a syringe having a plunger and a barrel, the syringe for storing the fluid to be added or the fluid to be removed;
a needle having a base and a tip, the base of the needle coupled to the barrel of the syringe, the needle further having a channel for carrying the fluid between the base and the tip; and
an electronic indicator coupled to an outside surface of the needle, such that when the tip of the needle contacts the conductive film of the tubing, the electronic indicator is activated.

2. The implant detection system of claim 1 wherein the electronic indicator is a light located outside of the patient.

3. The implant detection system of claim 1 further comprising: a signal processor configured to send an initial signal and measure a return signal, and wherein the electronic indicator is activated when the return signal is altered in response to the needle contacting the conductive film of the tubing.

4. The implant detection system of claim 1 further comprises a return electrode for testing the signal processor.

5. The implant detection system of claim 4 wherein the return electrode causes the electronic indicator to be activated when the needle contacts the return electrode.

6. The implant detection system of claim 1 wherein the needle includes a thin layer of insulation around a shank of the needle.

7. The implant detection system of claim 1 wherein the conductive film is a layer of gold.

8. The implant detection system of claim 7 wherein the conductive film is between 50 and 50,000 angstroms thick.

9. The implant detection system of claim 7 wherein the conductive film progressively increases in thickness along a length of the tubing from the gastric band to the access port.

10. The implant detection system of claim 1 wherein the initial signal is a biphasic, sinusoidal waveform.

11. The implant detection system of claim 1 wherein a length of the tubing allows for compatibility with MRI procedures.

12. The implant detection system of claim 3 wherein the signal generator comprises an electronic circuit having a comparator, wherein a first output of the comparator activates the electronic indicator and a second output of the comparator does not activate the electronic indicator.

13. An implant detecting apparatus that detects whether the implant detecting apparatus is in contact with an unintended portion of a gastric banding system for the treatment of obesity and/or obesity-related diseases, the implant detecting apparatus comprising:
a syringe for introducing or removing fluid from the gastric banding system;
a needle fluidly coupled to the syringe, the needle configured to contact a first portion of the gastric banding system or a second portion of the gastric banding system; and
an indicator having a warning light and coupled to the needle, the indicator measuring a first return signal and activating a warning light when the needle is in contact with the first portion of the gastric banding system, and measuring a second return signal and not activating the warning light when the needle is in contact with the second portion of the gastric banding system.

14. The implant detecting apparatus of claim 13 wherein the first portion of the gastric banding system is a tubing fluidly coupling an access port to an inflatable portion.

15. The implant detecting apparatus of claim 14 wherein the second portion of the gastric banding system is a septum of the access port.

16. The implant detecting apparatus of claim 14 wherein the first portion of the gastric banding system is a conductive metal integrated as the outer surface of the tubing.

17. A gastric banding system for the treatment of obesity and/or obesity-related diseases, the gastric banding system configured to alert a medical professional when a needle controlled by the medical professional contacts an unintended contact portion of the gastric banding system, the gastric banding system comprising:
a gastric band having an inflatable portion and disposed about the gastric-esophageal junction of the patient,
an access port having a septum for receiving the needle and allowing establishment of a fluid path between the needle and the inflatable portion of the gastric band, and
a tubing fluidly coupling the gastric band to the access port, the tubing serving as the unintended contact portion of the gastric banding system, the tubing having a conductive outer layer configured such that when conductive outer layer receives an electrical signal, the conductive outer layer alters and returns the electrical signal for activating a light exterior to a body of the patient.

18. The gastric banding system of claim 17 wherein the conductive outer layer is constructed out of a biocompatible material.

19. The gastric banding system of claim 18 wherein the biocompatible material is selected from a group consisting of a gold, a platinum, a titanium, a chromium, an indium-titanium oxide, a stainless steel, a tungsten, a nitinol, and combination thereof.

20. The gastric banding system of claim 17 wherein the conductive outer layer is between 50 and 50,000 angstroms thick, and wherein the conductive outer layer progressively increases in thickness along a length of the tubing from the gastric band to the access port.

21. An implant detecting apparatus that detects when the implant detecting apparatus is in contact with a conductive element disposed within a human body, the implant detecting apparatus comprising:
a syringe having a barrel for holding a fluid, a plunger to move within the barrel and to move the fluid into and out of the barrel, and a needle fluidly coupled to the barrel; and
an indicator electrically connected to the needle and having a warning light that is activated when the needle is in contact with the conductive element.

22. The implant detecting apparatus of claim 21 wherein the indicator is further configured to send a signal to the needle and measure a return signal from the needle to determine when the needle is in contact with the conductive element.
